(19) United States
(12) Patent Application Publication
(10) Pub. No.: US 2009/0317447 A1
(43) Pub. Date: Dec. 24, 2009

(54) Biodegradable bone graft for orthopedic use

(75) Inventors: Tzu-Yin Hsiao, Taipei City (TW); Yu-Te Lin, Anding Township (TW); Chien-Hsing Lin, Rende Township (TW); Hsiang-Yin Lu, Pintung City (TW)

Correspondence Address: BACON & THOMAS, PLLC, 625 SLATERS LANE, FOURTH FLOOR, ALEXANDRIA, VA, 22314-1176, US

(73) Assignee: SunMax Biotechnology Co., Ltd., Tainan County (TW)

(21) Appl. No.: 12/285,906

(22) Filed: Oct. 16, 2008

(30) Foreign Application Priority Data
Jun. 24, 2008 (TW) 097123492

(51) Int. Cl. A61F 2/00; A61F 2/28

(52) U.S. Cl. 424/426; 623/23.61

(57) ABSTRACT

In the present invention, a biodegradable bone graft is disclosed, which includes: a scaffold made of a biodegradable material; and a collagen-embedding matrix portion which completely encompasses the scaffold. The above-mentioned bone graft can increase the micro-porosity of the scaffold to enable cells to grow adhesively thereon. Compared with the scaffold only, the above-mentioned bone graft has high hydrophilicity. Hence, the bone graft of the present invention can efficiently retain tissue fluid, cell growth factors, blood and/or bone marrow which are mixed with the bone graft beforehand to achieve osteoinduction. Furthermore, the collagen-embedding matrix portion can also serve as a carrier to encompass other bone graft materials and drug molecules. The present invention also relates to a method for manufacturing the above-mentioned bone graft.

### BACKGROUND OF THE INVENTION

1. Field of the Invention

The present invention relates to a biodegradable bone graft and, more particularly, to a biodegradable bone graft for orthopedic use.

2. Description of Related Art

Traditionally, bone grafts or bone substitute materials for filling of bone defects are unable to induce bone regeneration and to completely patch those defects. Some researchers have proposed the use of bone grafts obtained from living humans to repair bone defects. Such bone grafts can be classified into three groups, namely autografts, homografts and heterografts. However, if autografts are used, additional surgery is required to take out the filling bones at another body place of the patient, leading to an increase in the number of wounds, thus possibly aggravating the patient's condition. If homografts or heterografts are applied, it is possible for immune rejection or viral infection to occur, thus causing problems with biocompatibility and patient safety.

When bone fillers made of natural or synthetic materials are temporarily implanted into animal bodies, they provide autologous cells with absorbable supports. As these supports decompose slowly, new tissues evolve. The above-mentioned concept gradually leads researchers to replace autografts with artificial bone grafts.

In general, desirable bone grafts for cell growth should: (1) be porous net structures beneficial for cell growth and deliveries of nutrients and metabolites; (2) be bio-compatible and bio-absorbable materials able to be controlled in their absorption rate; (3) have suitable surface chemical characteristics advantageous for cell attachment, proliferation, and differentiation; and (4) have the same physical property of the wound tissues.

Biodegradable materials such as artificial polymers can be a kind of biomedical materials capable of being applied in bone reconstruction. Nevertheless, when polymers such as polycaprolactone (PCL), polylactide (PLA), polyglycolide (PGA) and polydioxanone (PDO) are processed into scaffolds for osteocyte growth, these polymer scaffolds are too hydrophobic to adequately retain tissue fluid, cell growth factors, blood and/or bone marrow which are mixed with polymer scaffolds beforehand to achieve osteoinduction. Owing to incomplete attachment between polymer scaffolds and wound tissues, it is difficult for these polymer scaffolds to accomplish osteoconduction, i.e. that cells attach thereto and grow thereon. Therefore, if these polymers scaffolds are used for bone reconstruction, the aforesaid shortcomings should be overcome to achieve the goal of optimal osteo-reconstruction.

### SUMMARY OF THE INVENTION

The object of the present invention is to provide a biodegradable bone graft for orthopedic use to enable osteocytes to adhesively grow thereon. The above-mentioned bone graft has sufficient hydrophilicity, plasticity and flexibility. Hence, the above-mentioned bone graft can efficiently absorb tissue fluid and blood, and closely contact with soft or hard tissue. Once the above-mentioned bone graft is previously mixed with bone marrow, osteoconduction can be easily achieved. Furthermore, the above-mentioned bone graft can be a drug composite as a carrier for controlling the release of the drug.

To achieve the object, the present invention provides a biodegradable bone graft for orthopedic use. The biodegradable bone graft for orthopedic use comprises a scaffold made of a biodegradable material; and a collagen-embedding matrix portion which completely encompasses the scaffold.

The present invention also provides a method for preparing a biodegradable bone graft for orthopedic use, comprising the following steps: providing a scaffold made of a biodegradable material; preparing a collagen fibril paste; and forming a collagen-embedding matrix portion completely encompassing the scaffold by using the collagen fibril paste.

In the aforementioned method, the collagen-embedding matrix portion can be formed by the following steps comprising: pouring the collagen fibril paste into a predetermined container; putting the scaffold into the predetermined container; filling the predetermined container with the collagen fibril paste; and drying the collagen fibril paste.

Besides, the collagen concentration of the collagen fibril paste is preferably in the range of 10˜65 mg/mL, and more preferably in the range of 15˜45 mg/mL. If the collagen concentration is more than 65 mg/mL, the collagen fibril paste is too dense to wholly encompass the scaffold, and thus voids form easily. On the other hand, if the collagen concentration is less than 10 mg/ml, the collagen fibril paste is too diluted to bind with the scaffold, thereby being easy to loose apart from the scaffold after rehydration when clinical use.

In the foregoing biodegradable bone graft, the collagen in the collagen-embedding matrix portion can be at least one selected from the group consisting of type I collagen, type II collagen, and type III collagen. Also, the collagen in the collagen-embedding matrix portion can be acid-soluble collagen or acid-insoluble collagen. The skin thickness of the collagen-embedding matrix portion is preferably in the range of 0.5˜10 mm, and more preferably in the range of 13 mm. If the skin thickness of the collagen-embedding matrix portion is in the abovementioned range, the biodegradable bone graft can have sufficient hydrophilicity to absorb tissue fluid and blood.

Furthermore, the collagen-embedding matrix portion can further comprise a first additive which is hydroxyapatite (HA), tricalcium phosphate (TCP), HA/TCP composite, bioactive glass or the combination thereof. The ratio of the amount of the first additive to the collagen in the collagen-embedding matrix portion is preferably in the range of 5˜20:1, and more preferably in the range of 8˜15:1. If the first additive is added to the biodegradable bone graft in the range of the amount described above, it is sufficient to enhance osteoconduction.

In addition, the collagen-embedding matrix portion can further comprise a second additive which is bone morphogenetic protein, bone growth factor, antibiotic, drug or the combination thereof. Those skilled can decide the added amounts of various second additives according to the common sense in the art.

The biodegradable material can be at least one selected from the group consisting of polycaprolactone (PCL), polylactide (PLA), polyglycolide (PGA), poly(lactide-co-glycolide) (PLGA), and polydioxanone (PDO).

Preferably, the scaffold is a 2D or 3D micropore network. The scaffold can comprise a first portion and a second portion directly connecting with the first portion, and the cross-sectional area of the first portion is larger than that of the second portion. When the biodegradable bone graft contains the aforesaid scaffold, it can be applied as filler for burr holes of skull defects in the skull reconstruction. Since the burr holes can be easily filled with the second portion of the scaffold, and the first portion of the scaffold can prevent the whole bone graft from entry into the skull, the bone graft of the present invention has- high safety. Besides, the collagen of the bone graft has good adhesion to the skull. Thus, other processes of securing the bone graft are not required to prevent the departure of the bone graft from the skull, thereby saving operating time. Moreover, in addition to the shape illustrated above, the scaffold still can be sheet-shaped, pillar-shaped, cubic, conical, bar-shaped, or any shape demanded by clients. For example, if the bone graft contains the sheet-shaped scaffold, this bone graft can be applied to operations on thoracic and lumbar vertebra to enhance osteoconduction and to stabilize vertebra having shape-memory function.

In conclusion, the present invention uses the collagen-embedding matrix portion to completely encompass the biodegradable scaffold, and then produces the biodegradable bone graft used for orthopedics. Compared with the biodegradable scaffold only, the bone graft of the present invention has an improved hydrophilicity owing to the surface conformability and hydrophilicity of the encompassing collagen, and thus can overcome the drawback of poor contact of the scaffold with tissues.

Furthermore, the biocompatible and bio-absorbable collagen can promote cell proliferation and osteogenesis. Since the freeze-dried fiber network of the collagen is micro-porous, the bone graft of the present invention overwhelms the shortcoming of the scaffold having macropore network only which is unfavorable for cell attachment.

Moreover, the collagen in itself can be a vehicle for drug delivery. Hence, growth factors or other necessary drugs can be added in the collagen-embedding matrix portion.

Other objects, advantages, and novel features of the invention will become more apparent from the following detailed description when taken in conjunction with the accompanying drawings.

### BRIEF DESCRIPTION OF THE DRAWINGS

FIG. 1 is a perspective view of the biodegradable scaffold used in Examples 1 and 2 of the present invention;

FIG. 2 is a perspective view of the mold used in Examples 1 and 2 of the present invention;

FIGS. 3A to 3C show a flow chart for preparing biodegradable bone graft used for orthopedics in Examples 1 and 2 of the present invention;

FIG. 4A is a perspective view of the biodegradable bone graft prepared in Examples 1 and 2 of the present invention;

FIG. 4B is a perspective view of the biodegradable bone graft prepared in Example 3 of the present invention;

FIG. 5 is a perspective view of the biodegradable scaffold used in Example 4 of the present invention;

FIGS. 6A to 6C show a flow chart for preparing biodegradable bone graft used for orthopedics in Example 4 of the present invention;

FIG. 7A is a perspective view of the biodegradable bone graft prepared in Example 4 of the present invention; and

FIG. 7B is a perspective view of the biodegradable bone graft prepared in Example 5 of the present invention.

### DETAILED DESCRIPTION OF THE PREFERRED EMBODIMENT

Because of the specific embodiments illustrating the practice of the present invention, a person having ordinary skill in the art can easily understand other advantages and efficiency of the present invention through the content disclosed therein. The present invention can also be practiced or applied by other variant embodiments. Many other possible modifications and variations of any detail in the present specification based on different outlooks and applications can be made without departing from the spirit of the invention.

### Preparation of Biodegradable Scaffolds

Frozen compressed deposit manufacturing (FCDM) was used to produce a 3D scaffold. First, PCL material (SIGMA, No. 440744) was mixed with a solvent to prepare a proper concentration. PCL paste was injected by high-pressure gas from a nozzle (moving according to predetermined pathways of an x-y-z platform) to a cooled platform with low temperature, thereby congealing thereon. By this way, each plane of the PCL scaffold was constructed so that the 3D scaffold could be afforded.

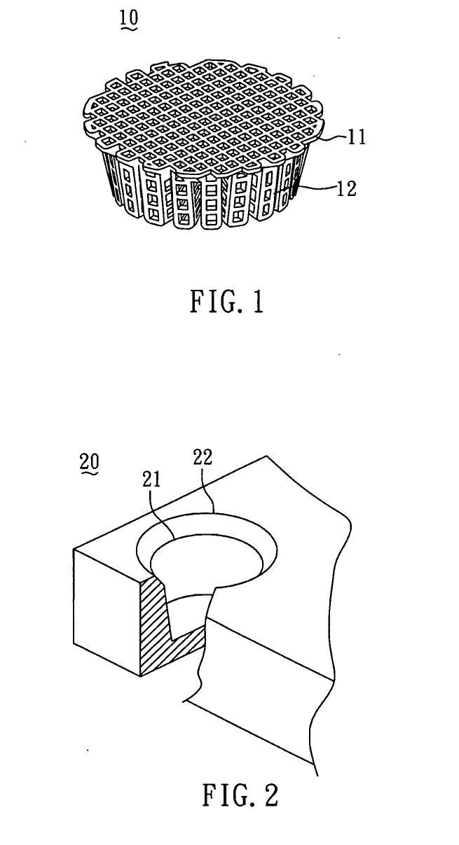

The scaffold prepared by the method described above is shown as FIG. 1. However, in addition to PCL, other suitable biodegradable materials can also be used. This scaffold 10 can comprise a first portion 11 and a second portion 12 directly connecting with the first portion 11, and the cross-sectional area of the first portion 11 is equal to or larger than that of the second portion 12. As shown in FIG. 1, the cross-sectional area of the first portion 11 reduces gradually until that is equal to the corresponding cross-sectional area of the second portion 12. In addition, the cross-sectional area of the end of the second portion 12 connnecting to the first portion 11 is larger than that of the other end of the second portion 12. It can be known that the second portion 12 is cone-shaped. Hence, when the biodegradable bone graft contains the aforesaid scaffold, it can be applied as filler for burr holes of skull defects in the skull reconstruction. Since the burr holes can be easily filled with the second portion of the scaffold, and the first portion of the scaffold can prevent the whole bone graft from entry into the skull, the bone graft of the present invention have high patient safety. Besides, the collagen of the bone graft has good adhesion to the skull. Thus, other processes of securing the bone graft are not required to prevent the departure of the skull, thereby saving operating time.

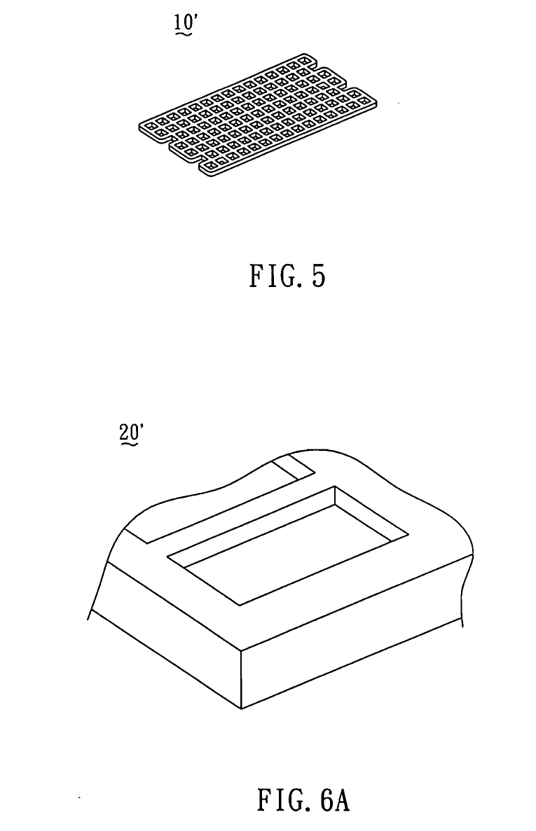

Besides, other-shaped scaffolds also can be fabricated by the aforesaid method. As shown in FIG. 5, there is a sheet-shaped scaffold 10′. If the bone graft is prepared by the sheet-shaped scaffold 10′, this bone graft can be applied to operations on thoracic and lumbar vertebra to enhance osteoconduction and to stabilize thoracic lumbar vertebra.

### Preparation of Collagen Fibers

The following is a method for fibrillation of collagen (conc.: 3 mg/mL, pH 2.0).

First, collagen (obtained by referring to the method disclosed in TW 236501) and 0.2 M phosphate buffered saline (PBS) were mixed with each other in the ratio of 9:1 (by weight or by volume). The collagen solution was stirred for 4 hours at 30±5° C. and the pH level of 7.0±0.2. Meanwhile, the collagen was reconstructed into collagen fibers. After centrifugation at 14,000 G for 1 hour, the collagen fibers were afforded with high concentration.

The aforesaid collagen fibers can be diluted with PBS to form a collagen fibril paste with proper collagen concentration. In general, the amount of the collagen in the collagen fibril paste can be in the range of 10˜65 mg/mL. Preferably, ceramic particles such as hydroxyapatite (HA), tricalcium phosphate (TCP), HA/TCP composite and so on, and/or bioactive glass can be selectively added in the collagen fibril paste to achieve osteoconduction.

For example, the ratio of those components by weight can be listed as follows.
1. The collagen fibers: HA/TCP composite=12:88; and
2. The collagen fibers: HA/TCP composite: bioactive glass=12:17.6:70.4.

The collagen fibril paste with ceramic particles in the abovementioned ratio can be prepared by: diluting the collagen fibers with PBS to form a collagen fibril paste with a predetermined collagen concentration; adding HA/TCP composite and bioactive glass to the collagen fibril paste; and stirring the collagen fibril paste by a stirring rod or by a stirrer.

### EXAMPLES 1 AND 2

The collagen fibers were diluted with PBS to form a collagen fibril paste with the collagen concentrations of 35 mg/mL (Example 1) and 65 mg/mL (Example 2).

In order to construct the collagen-embedding matrix portion with sufficient thickness to encompass the scaffold, the size of the predetermined mold should be larger than that of the scaffold. In general, the mold can be larger than the scaffold by about 0.5˜10 mm, but most preferably by 1˜5 mm. FIG. 2 shows the mold 20 used for the scaffold 10 shown in FIG. 1.

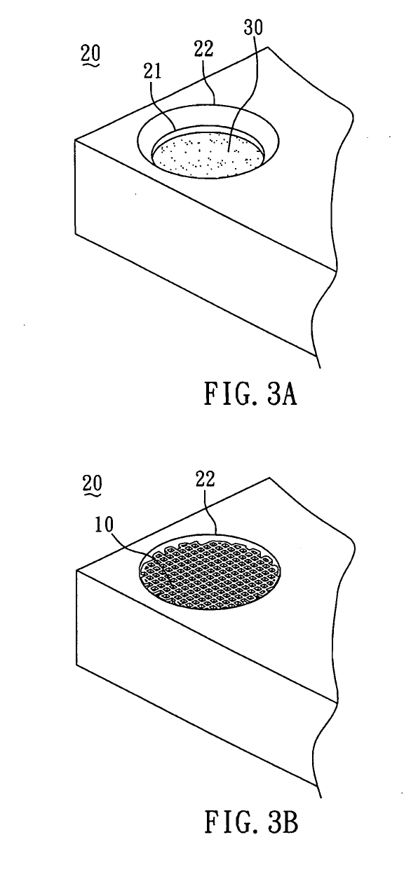

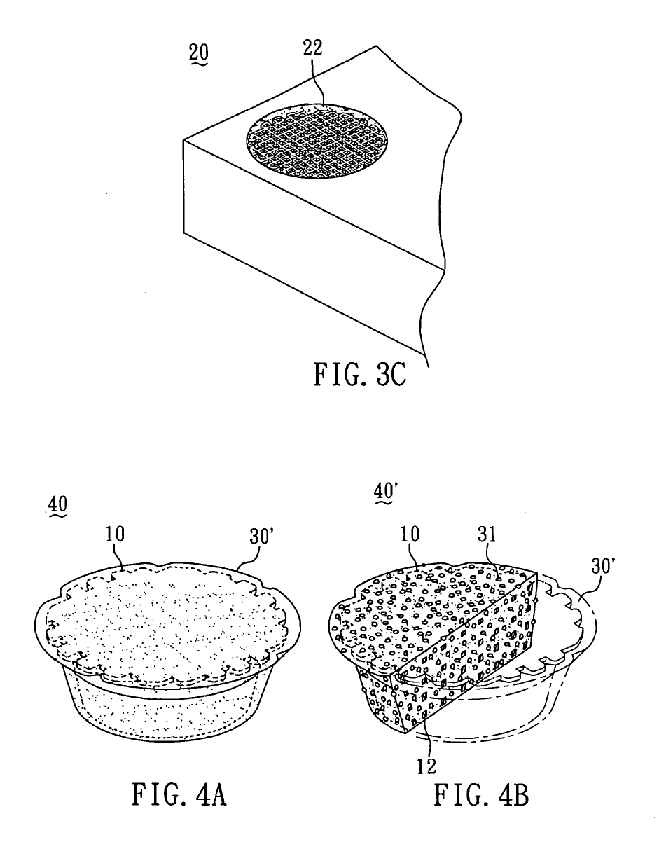

The method for preparing the biodegradable bone graft 40 of the present invention is described by the following. First, as shown in FIG. 3A, the collagen fibril paste 30 with adjusted concentration was poured into the mold 20 until the paste surface reached to a first plane 21 of the mold 20. Subsequently, as shown in FIG. 3B, the PCL scaffold 10 (FIG. 1) was put into the mold 20 filled with the collagen fibril paste 30. With reference to FIG. 3C, the collagen fibril paste 30 was poured into the mold 20 with the scaffold 10 therein until the surface of the collagen fibril paste 30 reached to a second plane 22 of the mold 20. In Examples 1 and 2, the total volume of the collagen fibril paste 30 poured into the mold 20 was approximately 1.5 mL.

After that, the collagen fibril paste 30 in the mold 20 was frozen at −60° C. for 12 hours, and was freeze-dried for 48 hours. Finally, the collagen-embedding matrix portion 30′, completely encompassing the scaffold 10, was formed. FIG. 4A shows the resultant bone graft 40 after the freeze-drying process.

### EXAMPLE 3

Except the collagen fibril paste 30 comprised a first additive 31 such as HA/TCP composite and bioactive glass, the bone graft of the present example was prepared in the manner substantially similar to Examples 1 and 2. In the collagen fibril paste 30 of the present invention, the ratio of the collagen to the first additive 31 is 12:88 by weight. FIG. 4B shows the resultant bone graft 40′ containing the first additive 31 after the freeze-drying process.

### EXAMPLE 4

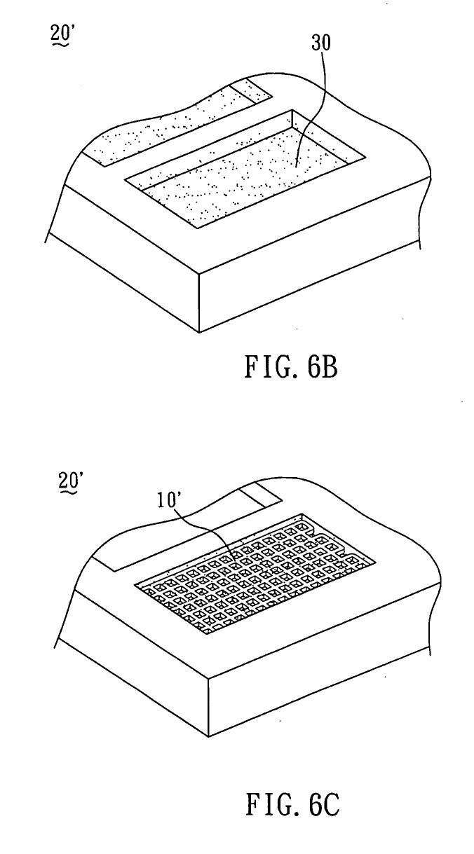

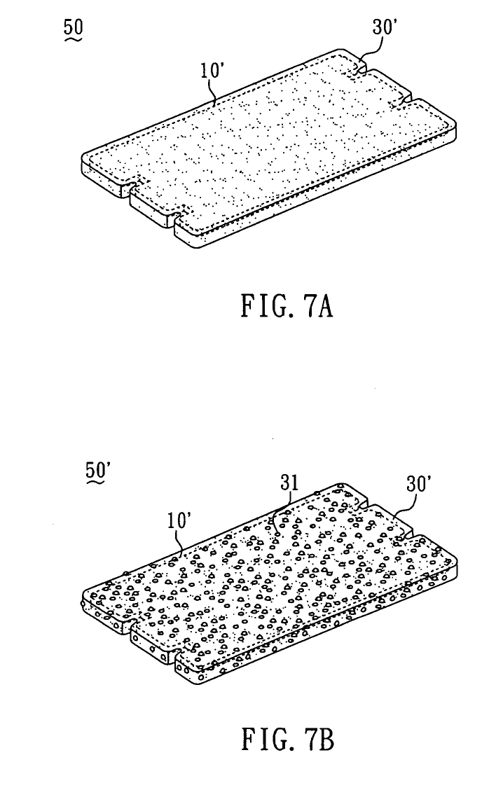

As shown in FIGS. 6A to 6C, except the sheet-shaped scaffold 10′ and the corresponding mold 20′ were used, the bone graft of the present example was prepared in the manner substantially similar to Examples 1 and 2. FIG. 7A shows the resultant bone graft 50 after the freeze-drying process.

### EXAMPLE 5

Except the sheet-shaped scaffold 10′ and the corresponding mold 20′ were used, the bone graft of the present example was prepared in the manner substantially similar to Example 3. FIG. 7B shows the resultant bone graft 50′ containing the first additive 31 after the freeze-drying process.

### COMPARATIVE EXAMPLE

In the present invention, the PCL scaffold 10 the same as used in Examples 1 and 2 was not encompassed by the collagen-embedding matrix portion 30′ formed from the collagen fibril paste 30, and was directly used as a bone graft.

### EXPERIMENTAL EXAMPLE

### Test Method:

a. The weights of the bone grafts 40 prepared in Examples 1 and 2 and the scaffold 10 in Comparative Example were respectively recorded before the bone grafts 40 and the scaffold 10 absorbed buffer.
b. The bone grafts 40 and the scaffold 10 were dipped in the PBS for 3 minutes to adequately absorb buffer.
c. The bone grafts 40 and the scaffold 10 were picked up with tweezers from PBS, and were put on a strainer for 30 seconds until there was no buffer dropped down. The weights of the bone grafts 40 and the scaffold 10 were recorded after buffer absorption.
d. Water absorption power of the bone grafts 40 and the scaffold 10 were evaluated by the following equation.

Liquid-holding capacity (%)=(Weight after buffer absorption−Weight before buffer absorption)/ Weight before buffer absorption×100%

TABLE 1

| | | | | Weight | |
|---|---|---|---|---|---|
| | | | Weight before | after | Liquid- |
| | Collagen fibril paste | | buffer | buffer | holding |
| | Conc. | Volume | absorption | absorption | capacity |
| | (mg/mL) | (mL) | (g) | (g) | (%) |
| Comparative | — | 0 | 0.5* | 0.695 | 39 |
| Example | | | | | |
| Example 1 | 35 | 1.5 | 0.561 | 1.089 | 94.2 |
| Example 2 | 65 | | 0.627 | 1.160 | 85 |

*The weight of the PCL scaffold was 0.5 g.

As listed in Table 1, when the PCL scaffold 10 is encompassed by the collagen-embedding matrix portion 30′ formed from the collagen fibril paste 30, the liquid-holding capacity of the bone grafts 40 dramatically increases by at least 46% (shown as 65 mg/mL). However, the liquid-holding capacity of the bone grafts 40 decreases as the collagen concentration of the collagen fibril paste 30 increases.

In fixed volume, pores formed between adjacent collagen fibers at high collagen concentration are definitely smaller than those at low collagen concentration. In fact, collagen holds water molecules by trapping them into the pores. If the collagen matrix has more and/or larger pores than another, this collagen matrix relatively has a higher water-holding capacity. Therefore, it is difficult for an excessively dense collagen solution to encompass the scaffold, to enter pores of the scaffold, and to mix well with additives.

Besides, the collagen amount of the collagen-embedding matrix portion also influences the strength of the water-absorbed bone graft. If the concentration of collagen is extremely low, the water-absorbed collagen-embedding matrix portion is too loose to bind well with the PCL scaffold even though the bone graft has good water-holding capacity. Hence, the concentration of collagen also can not be too low.

In conclusion, compared with conventional bone grafts made only of PCL or PLA, the present invention uses the collagen-embedding matrix portion to encompass the scaffold to form the biodegradable bone graft for orthopedics. The present invention can improve the hydrophobicity of the PCL scaffold to produce the bone graft having advanced hydrophilicity. Therefore, the bone graft of the present invention is advantageous to tissue recovery.

Although the present invention has been explained in relation to its preferred embodiment, it is to be understood that many other possible modifications and variations can be made without departing from the scope of the invention as hereinafter claimed.

What is claimed is:

1. A biodegradable bone graft for orthopedic use comprising:
a scaffold made of a biodegradable material; and
a collagen-embedding matrix portion which completely encompasses the scaffold.

2. The biodegradable bone graft for orthopedic use as claimed in claim 1, wherein the collagen in the collagen-embedding matrix portion is at least one selected from the group consisting of type I collagen, type II collagen, and type III collagen.

3. The biodegradable bone graft for orthopedic use as claimed in claim 1, wherein the collagen in the collagen-embedding matrix portion is acid-soluble collagen, or acid-insoluble collagen.

4. The biodegradable bone graft for orthopedic use as claimed in claim 1, wherein the collagen-embedding matrix portion further comprises a first additive which is at least one selected from the group consisting of hydroxyapatite (HA), tricalcium phosphate (TCP), HA/TCP composite, bioactive glass, and the combination thereof.

5. The biodegradable bone graft for orthopedic use as claimed in claim 4, wherein the ratio of the amount of the first additive to the collagen in the collagen-embedding matrix portion is 5˜20:1.

6. The biodegradable bone graft for orthopedic use as claimed in claim 1, wherein the collagen-embedding matrix portion further comprises a second additive which is at least one selected from the group consisting of bone morphogenetic protein, bone growth factor, antibiotic, drug, and the combination thereof.

7. The biodegradable bone graft for orthopedic use as claimed in claim 1, wherein the biodegradable material is at least one selected from the group consisting of polycaprolactone (PCL), polylactide (PLA), polyglycolide (PGA), poly(lactide-co-glycolide) (PLGA), and polydioxanone (PDO).

8. The biodegradable bone graft for orthopedic use as claimed in claim 1, wherein the scaffold is a 2D or 3D micropore network.

9. The biodegradable bone graft for orthopedic use as claimed in claim 1, wherein the collagen-embedding matrix portion has the skin thickness of 0.5˜10 mm.

10. The biodegradable bone graft for orthopedic use as claimed in claim 1, wherein the scaffold comprises a first portion and a second portion directly connecting the first portion, and the cross-sectional area of the first portion is larger than that of the second portion.

11. The biodegradable bone graft for orthopedic use as claimed in claim 1, wherein the scaffold is sheet-shaped, pillar-shaped, cubic, conical, or bar-shaped.

12. A method for preparing a biodegradable bone graft for orthopedic use, comprising the following steps:
providing a scaffold made of a biodegradable material;
preparing a collagen fibril paste; and
forming a collagen-embedding matrix portion completely encompassing the scaffold by using the collagen fibril paste.

13. The method as claimed in claim 12, wherein the step of forming the collagen-embedding matrix portion comprises the following substeps:
pouring the collagen fibril paste into a predetermined container;
putting the scaffold into the predetermined container;
filling the predetermined container with the collagen fibril paste; and
drying the collagen fibril paste.

14. The method as claimed in claim 12, wherein the collagen concentration of the collagen fibril paste is 10˜65 mg/mL.

15. The method as claimed in claim 12, wherein the collagen in the collagen fibril paste is at least one selected from the group consisting of type I collagen, type II collagen, and type III collagen.

16. The method as claimed in claim 12, wherein the collagen fibril paste further comprises a first additive which is at least one selected from the group consisting of hydroxyapatite (HA), tricalcium phosphate (TCP), HA/TCP composite, bioactive glass, and the combination thereof.

17. The method as claimed in claim 16, wherein the ratio of the amount of the first additive to the collagen in the collagen fibril paste is 5˜20:1.

18. The method as claimed in claim 12, wherein the collagen fibril paste further comprises a second additive which is at least one selected from the group consisting of bone morphogenetic protein, bone growth factor, antibiotic, drug, and the combination thereof.

19. The method as claimed in claim 12, wherein the biodegradable material is at least one selected from the group consisting of polycaprolactone (PCL), polylactide (PLA), polyglycolide (PGA), poly(lactide-co-glycolide) (PLGA) and polydioxanone (PDO).

20. The method as claimed in claim 12, wherein the scaffold is a 2D or 3D micropore network.

21. The method as claimed in claim 12, wherein the collagen-embedding matrix portion has the skin thickness of 0.5˜10 mm.
